(19) United States
(12) Patent Application Publication
(10) Pub. No.: US 2023/0192596 A1
(43) Pub. Date: Jun. 22, 2023

(54) METHOD FOR PRODUCING ACETAMINOPHEN

(71) Applicant: API CORPORATION, Chikujo-gun (JP)

(72) Inventors: Masato MURAI, Chikujo-gun (JP); Hirotsugu TANIIKE, Chikujo-gun (JP); Yurie KOBA, Chikujo-gun (JP)

(73) Assignee: API CORPORATION, Chikujo-gun (JP)

(21) Appl. No.: 17/999,152

(22) PCT Filed: May 14, 2021
(86) PCT No.: PCT/JP2021/018363

§ 371 Date: Nov. 17, 2022

(30) Foreign Application Priority Data
May 18, 2020 (JP) 2020-086766

(51) Int. Cl. C07C 231/10; B01J 23/44; B01J 31/06; B01J 35/02; B01J 35/10; B01J 35/00

(52) U.S. Cl. CPC C07C 231/10; B01J 23/44; B01J 31/06; B01J 35/026; B01J 35/1038; B01J 35/1042; B01J 35/1047; B01J 35/1019; B01J 35/1023; B01J 35/1028; B01J 35/1061; B01J 35/0066

(57) ABSTRACT

A method for producing acetaminophen may include causing p-nitrophenol to undergo an acetamination reaction to produce the acetaminophen, by passing a solution containing the p-nitrophenol through a column packed with a catalyst while also passing an acetylating agent and hydrogen through the column. The catalyst may be a supported metal catalyst in which a metal element is supported on a synthetic adsorbent, and a reaction temperature of the acetamination reaction is 0° C. to 60° C., and a reaction pressure of the acetamination reaction is 0.1 MPa to 1 MPa. With the method, it is possible to continuously produce acetaminophen safely and inexpensively with high selectivity and good yield, at a low reaction temperature and a low reaction pressure.

### TECHNICAL FIELD

The present invention relates to a method for producing acetaminophen, which is useful as a medicine. Background Art

Acetaminophen is an antipyretic and analgesic drug that has been widely used for a long time. Acetaminophen is a safe drug that can be administered not only to adults but also to children.

In the related art, known methods for producing acetaminophen include batch reaction methods. For example, one known method is a method in which p-nitrophenol, acetic acid, and a metal catalyst are added to a reaction vessel, hydrogen is added thereto, and a reaction is caused to take place at a high temperature to produce acetaminophen (Patent Literature 1).

Unfortunately, in the method of Patent Literature 1, since the reaction temperature is high, and intense heat generation occurs when the catalyst is added, control of the reaction is difficult.

Accordingly, there is a need for an industrial production method that is safer and highly productive.

A method that increases productivity is a continuous reaction method.

For example, a method is known in which p-nitrophenol is added to an acetic anhydride/acetic acid solution to form a solution, and the solution is passed through a column packed with a noble metal catalyst, specifically, a Pd/C catalyst, to cause the p-nitrophenol to undergo a reaction at a hydrogen pressure of 8 MPa to 10 MPa and a reaction temperature of 90 to 140° C., thereby continuously producing acetaminophen (Patent Literature 2).

Unfortunately, in the method of Patent Literature 2, equipment that can withstand very high pressure conditions is required, and the reaction temperature is high. In addition, in instances where the reaction takes place continuously at a high temperature and a high pressure for a long time, there is a possibility that early degradation of the catalyst may occur.

Accordingly, there is a need for a production method that is a continuous production method that enables reactions to take place under milder conditions, conserves energy, and can be implemented at low costs, with the costs including, for example, equipment cost.

PTL 1: WO 2017/154024 A1

PTL 2: CN 102060729 A

### SUMMARY OF INVENTION

An object of the present invention is to provide a method for continuously producing acetaminophen safely and inexpensively with high selectivity and good yield, at a low reaction temperature and a low reaction pressure.

The present inventors discovered that acetaminophen can be produced safely and inexpensively with high selectivity and good yield, even at a low reaction pressure and a low reaction temperature, by causing p-nitrophenol to undergo a reaction by continuously passing a solution containing the p-nitrophenol through a column packed with a catalyst in which a metal element is supported on a synthetic adsorbent, while also continuously passing an acetylating agent and hydrogen through the column.

Features of the present invention are as follows.

[1] A method for producing acetaminophen, the method comprising causing p-nitrophenol to undergo an acetamination reaction to produce the acetaminophen, by passing a solution containing the p-nitrophenol through a column packed with a catalyst while also passing an acetylating agent and hydrogen through the column, wherein

the catalyst is a supported metal catalyst in which a metal element is supported on a synthetic adsorbent, and

a reaction temperature of the acetamination reaction is 0° C. to 60° C., and a reaction pressure of the acetamination reaction is 0.1 MPa to 1 MPa.

[2] The method for producing acetaminophen according to [1], wherein the synthetic adsorbent is a styrene-divinylbenzene-based copolymer.

[3] The method for producing acetaminophen according to [1] or [2], wherein the styrene-divinylbenzene-based copolymer is a styrene-divinylbenzene copolymer, and the metal element is palladium and/or platinum.

[4] The method for producing acetaminophen according to any one of [1] to [3], wherein the synthetic adsorbent is a porous synthetic adsorbent having a pore volume of 0.1 mL/g to 3.0 mL/g.

[5] The method for producing acetaminophen according to any one of [1] to [4], wherein the synthetic adsorbent is a porous synthetic adsorbent having a BET specific surface area of 200 m²/g to 2000 m²/g.

[6] The method for producing acetaminophen according to any one of [1] to [5], wherein the synthetic adsorbent is a porous synthetic adsorbent having a mode pore radius of 1 nm to 50 nm.

[7] The method for producing acetaminophen according to any one of [1] to [6], wherein an amount of the supported metal element in the supported metal catalyst is 1 mass % to 25 mass % based on a mass of the supported metal catalyst. Advantageous Effects of Invention

With the method of the present invention for producing acetaminophen, it is possible to continuously produce acetaminophen safely and inexpensively with high selectivity and good yield, at a low reaction temperature and a low reaction pressure.

### BRIEF DESCRIPTION OF DRAWINGS

FIG. 1 is a system diagram of a flow synthesis system, illustrating an exemplary embodiment of a method of the present invention for producing acetaminophen.

FIG. 2 is a system diagram of a flow synthesis system including a back-pressure valve, illustrating another exemplary embodiment of the method of the present invention for producing acetaminophen.

### DESCRIPTION OF EMBODIMENTS

The present invention will be described in detail below.

### [Method for Producing Acetaminophen]

The method for producing acetaminophen of the present invention comprises causing p-nitrophenol to undergo an acetamination reaction to produce the acetaminophen, by passing a solution containing the p-nitrophenol (This solution may be referred to as “p-nitrophenol solution” hereinafter.) through a column packed with a catalyst while also passing an acetylating agent and hydrogen through the column, (This step may be referred to as “acetamination step of the present invention” hereinafter.) wherein the catalyst is a supported metal catalyst in which a metal element is supported on a synthetic adsorbent (The supported metal catalyst may be referred to as “supported metal catalyst of the present invention” hereinafter.), and a reaction temperature of the acetamination reaction is 0° C. to 60° C., and a reaction pressure of the acetamination reaction is 0.1 MPa to 1 MPa.

### <Acetamination Step>

Methods for implementing the acetamination step of the present invention are not particularly limited.

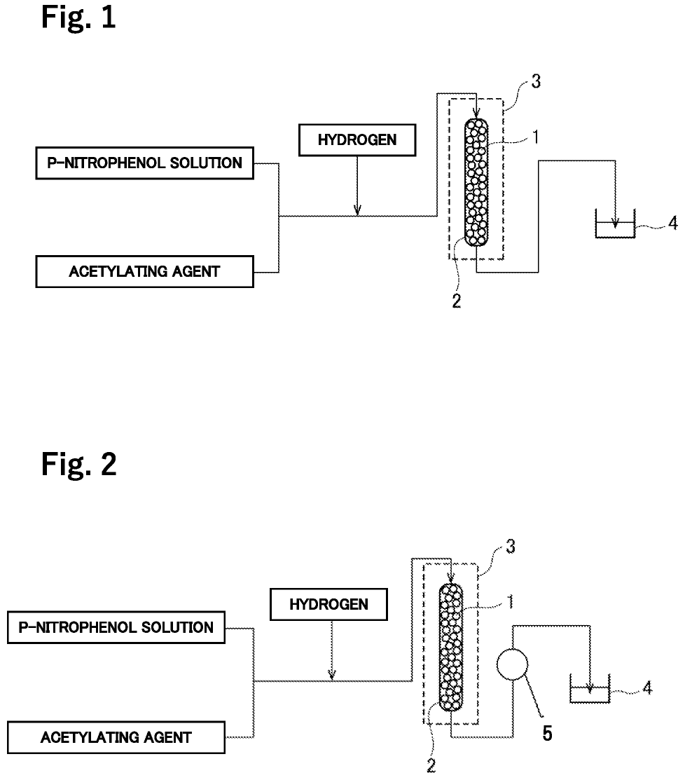

An exemplary method is a method that uses a flow synthesis system, as illustrated in FIGS. 1 and 2. In the method, a p-nitrophenol solution is continuously passed through a reaction vessel 3, which includes a column 2 packed with a supported metal catalyst 1 of the present invention, while an acetylating agent and hydrogen are also continuously passed through the reaction vessel 3, to cause the p-nitrophenol to continuously undergo an acetamination reaction with the acetylating agent and the hydrogen in the presence of the supported metal catalyst of the present invention within the column 2, and a reaction product liquid containing acetaminophen, which flows from the column 2, is received in a collection reservoir 4.

The flow synthesis system of FIG. 2 has a similar configuration to that of the flow synthesis system of FIG. 1, with a difference being that a back-pressure valve 5 is provided in a flow path through which the reaction product liquid coming from the reaction vessel 3 is delivered to the collection reservoir 4.

This flow synthesis system will be described later.

### <P-Nitrophenol Solution>

The p-nitrophenol, which is a raw material for the production of acetaminophen, may be a commercially available product or one prepared in accordance with a known method.

The solvent for use in the p-nitrophenol solution is not particularly limited as long as the solvent can dissolve p-nitrophenol and does not retard the progress of the reaction. Examples of the solvent include alcohol solvents and carboxylic acid solvents. Examples of the alcohol include methanol, ethanol, and propanol, and examples of the carboxylic acid include formic acid, acetic acid, and propionic acid. Methanol and acetic acid are preferable from the standpoint of cost, reactivity, and the like.

One of these solvents may be used alone, or two or more thereof may be combined in any combination in any ratio and used.

A concentration of the p-nitrophenol in the p-nitrophenol solution is not particularly limited as long as the flow thereof into the column is not hindered. The concentration of the p-nitrophenol in the p-nitrophenol solution may be specified from the standpoint of productivity and reactivity and is typically 0.1 mass % to 80 mass %, preferably 10 mass % to 70 mass %, and particularly preferably 20 mass % to 60 mass %.

### <Hydrogen>

An amount of use of the hydrogen (hydrogen gas) is not particularly limited as long as the reaction can proceed. The amount of use of the hydrogen (hydrogen gas) is typically greater than or equal to 1 mol and preferably greater than or equal to 3 mol and is typically less than or equal to 20 mol and preferably less than or equal to 10 mol, per mol of the p-nitrophenol.

Methods for feeding the hydrogen are not particularly limited. The hydrogen may be continuously introduced into and mixed with the p-nitrophenol solution or a p-nitrophenol solution that contains an acetylating agent, in a flow path upstream of the column 2, or the hydrogen may be directly injected into the column 2. The hydrogen may be used by being partially or entirely dissolved in the solvent of the p-nitrophenol solution.

The hydrogen may be used by being mixed with an inert gas, such as nitrogen, helium, or argon.

### <Acetylating Agent>

The acetylating agent is not particularly limited as long as the acetylating agent can acetylate amino groups. Typically, the acetylating agent to be used is one or more of acetylating agents such as acetic anhydride, acetyl chloride, and the like. Acetic anhydride is preferable from the standpoint of cost and reactivity.

An amount of use of the acetylating agent is not particularly limited. The amount of use of the acetylating agent may be specified from the standpoint of reactivity and is typically 1 mol to 10 mol, preferably 1 mol to 5 mol, and more preferably 1 mol to 2 mol, per mol of the p-nitrophenol.

The acetylating agent may be premixed with the solution containing p-nitrophenol, may be continuously mixed with the p-nitrophenol solution by introducing the acetylating agent into at least one of the p-nitrophenol solution delivery flow paths upstream and downstream of the column 2, or may be introduced into the column 2 separately from the p-nitrophenol solution and continuously mixed with the p-nitrophenol solution within the column 2. From the standpoint of rapidly converting an unstable intermediate product into a target product, it is preferable that the acetylating agent be continuously mixed with the p-nitrophenol solution in the flow path upstream of the column 2.

### <Supported Metal Catalyst>

The supported metal catalyst of the present invention is a catalyst in which a metal is fixed, with the metal element being supported on a synthetic adsorbent.

The metal element that can be used in the supported metal catalyst of the present invention is not particularly limited as long as the metal element has activity for reducing nitro groups. Typically, the metal element may be palladium (Pd), platinum (Pt), rhodium (Rh), ruthenium (Ru), silver (Ag), or a mixture of two or more of these. Among these metal elements, Pd alone and a mixture of Pd and at least one selected from Pt, Rh, Ru, and Ag are preferable. From the standpoint of catalytic performance, Pd and/or Pt are preferable, and Pd alone is particularly preferable.

An amount of the supported metal element may be specified from the standpoint of catalytic performance and cost. The lower limit of a content of the metal element in the supported metal catalyst of the present invention is typically greater than or equal to 0.1 mass %, preferably greater than or equal to 1 mass %, more preferably greater than or equal to 3 mass %, and particularly preferably greater than or equal to 5 mass %, and the upper limit of the content is typically less than or equal to 25 mass %, preferably less than or equal to 20 mass %, more preferably less than or equal to 15 mass %, and particularly preferably less than or equal to 10 mass %.

In the present invention, the synthetic adsorbent is a porous synthetic adsorbent formed of a porous organic polymer produced by chemical synthesis.

Examples of the synthetic adsorbent for use in the present invention include aromatic, substituted aromatic, or acrylic polymers or copolymers (hereinafter, a “polymer or copolymer” may be referred to as a “(co)polymer”).

Examples of the aromatic (co)polymers include styrene-divinylbenzene copolymers and divinylbenzene polymers.

Examples of the substituted aromatic (co)polymers include bromostyrene-divinylbenzene copolymers.

Examples of the acrylic (co)polymers include methacrylic acid ester-based (co)polymers, such as methyl methacrylate-ethylene glycol bis(methacrylate) copolymers.

Among these, aromatic (co)polymers are preferable, styrene-divinylbenzene-based copolymers, such as styrene-divinylbenzene copolymers and bromostyrene-divinylbenzene copolymers, are more preferable, and styrene-divinylbenzene copolymers are particularly preferable. These copolymers have a crosslinked structure insoluble in organic solvents and are, therefore, stable even in acidic or alkaline solutions.

It is preferable, in terms of reduced influence on the reaction, that the synthetic adsorbent for use in the present invention be a non-polar adsorbent or an adsorbent substantially free of functional groups such as an ion-exchange group, which may be, for example, an adsorbent having an ion exchange capacity of less than 1 meq/g.

The porous synthetic adsorbent for use in the present invention typically has a pore volume of 0.1 mL/g to 3 mL/g so that reactivity can be improved. The pore volume is preferably 0.5 mL/g to 2 mL/g and particularly preferably 1 mL/mL/g to 1.5 mL/g.

The porous synthetic adsorbent typically has a BET specific surface area of 200 m²/g to 2000 m²/g so that reactivity can be improved. The BET specific surface area is preferably 300 m²/g to 1500 m²/g, more preferably 400 m²/g to 1000 m²/g, and particularly preferably 500 m²/g to 700 m²/g.

The porous synthetic adsorbent typically has a mode pore radius of 1 nm to 50 nm so that reactivity can be improved. The mode pore radius is preferably 5 nm to 40 nm and particularly preferably 10 nm to 30 nm.

It is preferable that the synthetic adsorbent for use in the present invention be a porous synthetic adsorbent having relatively large pores.

In the present invention, the pore volume, the BET specific surface area, and the mode pore radius of the porous synthetic adsorbent can be measured with a nitrogen gas adsorption method in accordance with a common procedure.

The synthetic adsorbent may have a shape and a size that are not particularly limited, as long as the synthetic adsorbent can be packed into the column and does not hinder the flow of the reaction liquid. The synthetic adsorbent that can be used may be in the form of particles, pellets, a film, or a cylinder. From the standpoint of ease of packing, it is more preferable that the synthetic adsorbent be in the form of particles.

The synthetic adsorbent in the form of particles has a particle size that is typically within a range of 1 μm to 2000 μm and preferably within a range of 3 μm to 2000 μm. From the standpoint of ease of industrial handling and the like, it is preferable that the particle size of the synthetic adsorbent be within a range of 4 μm to 1000 μm, and a mode particle size thereof be greater than or equal to 50 μm. The mode particle size is preferably greater than or equal to 150 μm and particularly preferably greater than or equal to 250 μm.

The particle size of the synthetic adsorbent is an average particle size measured with a laser diffraction particle size distribution measurement method in accordance with a common procedure.

The synthetic adsorbent for use in the present invention may be a commercially available product, examples of which include DIAION HP20SS, HP20, and HP21 and SEPABEADS SP20SS, manufactured by Mitsubishi Chemical Corporation (“DIAION” and “SEPABEADS” are registered trademarks); and Amberlite XAD™-2, XAD™4, and XAD™7HP, manufactured by Organo Corporation (“Amberlite” is a registered trademark). Among these, HP20SS, HP20, and SP20SS are preferable from the standpoint of reactivity.

Details of these commercially available synthetic adsorbents are shown in Table 1 below.

TABLE 1

| | | BET | | Mode | Mode |
|---|---|---|---|---|---|
| | | specific | Pore | pore | particle |
| Product | | surface | volume | radius | size |
| name | Structure | area [m²/g] | [mL/g] | [nm] | [μm] |
| SEPABEADS | Styrene- | 560 | 1.2 | 29 | 63 or |
| SP20SS | divinyl- | | | | more |
| | benzene | | | | |
| | copolymer | | | | |
| DIAION | Styrene- | 560 | 1.2 | 29 | 63 or |
| HP20SS | divinyl- | | | | more |
| | benzene | | | | |
| | copolymer | | | | |
| DIAION | Styrene- | 590 | 1.3 | 29 | 250 or |
| HP20 | divinyl- | | | | more |
| | benzene | | | | |
| | copolymer | | | | |
| DIAION | Styrene- | 640 | 1.3 | 11 | 250 or |
| HP21 | divinyl- | | | | more |
| | benzene | | | | |
| | copolymer | | | | |
| Amberlite | Styrene- | 880 | 1.3 | 12 | 490 or |
| XAD ™4 | divinyl- | | | | more |
| | benzene | | | | |
| | copolymer | | | | |
| Amberlite | Acrylic | 500 | 0.6 | 10 | 430 or |
| XAD ™7HP | polymer | | | | more |

Preferably, the supported metal catalyst of the present invention is a catalyst in which Pd is supported on a synthetic adsorbent formed of a styrene-divinylbenzene copolymer (which may hereinafter be referred to as “Pd/PS-DVB”) or a catalyst in which Pt is supported on a synthetic adsorbent formed of a styrene-divinylbenzene copolymer (which may hereinafter be referred to as “Pt/PS-DVB”). Particularly preferably, the supported metal catalyst is Pd/PS-DVB.

With the use of the supported metal catalyst of the present invention, it is possible to produce acetaminophen highly efficiently, safely, and inexpensively with high selectivity and good yield, even at a low pressure and a low temperature.

The supported metal catalyst of the present invention can be produced with a method known in the art, such as the method described in JP 2008-114164 A. For example, the supported metal catalyst can be produced as follows. The synthetic adsorbent and a metal salt are added to an organic solvent and thoroughly stirred. Subsequently, the resulting synthetic adsorbent on which the metal salt has been adsorbed is collected by filtration, washed with water and methanol, and dried.

### <Flow Synthesis System>

The flow synthesis system, which is suitable for the implementation of the method of the present invention for producing acetaminophen, is a system that uses a reaction vessel having an inlet and an outlet and simultaneously carries out the addition of raw materials through the inlet, the reaction, and the collection of the formed product from the outlet. The concept of the flow synthesis system is well known to those skilled in the art (e.g., “Flow-Micro Synthesis”, published by Kagaku Dojin in 2014, page 9). In the flow synthesis system, the column into which the supported metal catalyst of the present invention is packed is in the form of a narrow pipe.

The material of the column associated with the present invention is not particularly limited. Examples of the material of the column include glass, stainless steel (SUS), Hastelloy, and Teflon (registered trademark).

The column may have a size that is not particularly limited as long as the size is suitable for the reaction. Examples of columns that may be used include columns having a size of 10 mm (diameter)×100 mm (length) and columns having a size of 10 mm (diameter)×250 mm (length).

Examples of catalyst-packed columns include one in which Pd/PS-DVB (Pd: 2.55 g, 0.9 mmol/g, styrene-divinylbenzene copolymer: DIAION HP20, manufactured by Mitsubishi Chemical Corporation (“DIAION” is a registered trademark)) is close-packed into an SUS column (10 mm×100 mm) and one in which Pd/PS-DVB (Pd: 2.55 g, 0.9 mmol/g, styrene-divinylbenzene copolymer: DIAION HP20, manufactured by Mitsubishi Chemical Corporation (“DIAION” is a registered trademark)) is close-packed into an SUS column (10 mm×250 mm).

A tube that is used as the flow path for introducing and discharging a substrate and the like into and from the column is not particularly limited. Specific examples of the tube include a Teflon tube having an inside diameter of 1 mm (“Teflon” is a registered trademark).

The introduction and discharge of the substrate and the like into the column can be carried out by delivering the liquid with a syringe pump, a diaphragm pump, a mass flow controller, and the like.

A back-pressure valve and an in-line analyzer may be provided in the flow path on the side to which the reaction product liquid from the column is discharged.

### <Reaction Conditions>

The reaction temperature of the acetamination reaction of the present invention is a temperature of the outside of the column packed with the supported metal catalyst of the present invention. The reaction temperature may be specified from the standpoint of reactivity, productivity, and the like and is typically 0° C. to 60° C., preferably 5° C. to 50° C., and particularly preferably 10° C. to 40° C. When the reaction temperature is less than any of the lower limits, the reactivity may decrease. When the reaction temperature is greater than any of the upper limits, a side reaction may cause a decrease in the yield and purity and degradation in the supported metal catalyst of the present invention.

The lower limit of a reaction pressure of the acetamination reaction of the present invention is typically greater than or equal to 0.1 MPa and preferably greater than or equal to 0.2 MPa, and the upper limit thereof is typically less than or equal to 1 MPa, preferably less than or equal to 0.8 MPa, and particularly preferably less than or equal to 0.6 MPa. In instances where the reaction is performed at a reaction pressure within any of these ranges, a hydrogen concentration of the p-nitrophenol solution increases, which enables the reaction to proceed efficiently.

The reaction pressure can be adjusted by applying a back pressure with a back-pressure valve or the like to the flow path downstream of the column packed with the supported metal catalyst of the present invention.

A reaction time of the acetamination reaction of the present invention is the time (retention time) during which the reaction liquid remains within the column packed with the supported metal catalyst of the present invention. The reaction time is typically 0.1 seconds to 60 seconds and preferably 0.1 seconds to 30 seconds, depending on the reaction temperature and the reaction pressure.

### <Post-Treatment>

The isolation of acetaminophen, which is the target product, from the reaction product liquid obtained in the acetamination step of the present invention, may be carried out by performing, on the reaction product liquid, a process such as neutralization, liquid-phase separation, condensation, or filtration or by using a known purification method, such as crystallization or column chromatography.

### EXAMPLES

The present invention will be described in more detail with reference to examples. The scope of the present invention is not limited to the examples described below.

In the following Examples and Comparative Examples, a ratio between the feed rates (mL/minute) of p-nitrophenol, acetic anhydride, and a hydrogen gas is 1:0.9:67 unless otherwise specified. The reaction time is the time during which the mixture liquid remains within the column.

### [Abbreviations]

In the examples, abbreviations each represent a compound, as listed below.

PAP: p-aminophenol

APAP: acetaminophen

PAAPA: 4-acetamidophenyl acetate

PNP: p-nitrophenol

PNPA: 4-nitrophenyl acetate

MeOH: methanol

AcOH: acetic acid

### [Flow Synthesis Apparatus]

The following flow synthesis apparatus was used in the Examples and Comparative Examples, described below.

Asia Flow Chemistry System, manufactured by Syriss Ltd.

### [Analysis Method 1 (HPLC)]

The apparatus and conditions used for the analysis of the reaction product liquid in the Examples and Comparative Examples, described below, are as shown in Table 2 below.

TABLE 2

| Analyzer | Agilent 1100, manufactured by Agilent | | |
|---|---|---|---|
| Column | Unison UK-C18 | | |
| | 3 μm, 4.6 mm I.D. × 250 mm | | |
| Mobile | 0.1 vol % aqueous phosphoric acid solution | | |
| phase A | | | |
| Mobile | Acetonitrile | | |
| phase B | | | |
| | Time | Mobile | Mobile |
| | (min) | phase A (%) | phase B (%) |
| Gradient | 0 | 90 | 10 |
| | 0 to 15 | 90→20 | 10→80 |

| | |
|---|---|
| Flow rate | 1.0 mL/min |
| Detection | 240 nm |
| wavelength | |
| Column | 40° C. |
| temperature | |
| Analysis | 15 min |
| time | |
| Retention | PAP: 2.7 min |
| time | APAP: 6.3 min |
| | PAAPA: 9.7 min |
| | PNP: 11.5 min |
| | PNPA: 13.8 min |

### Synthesis Example 1

A supported metal catalyst was produced with a synthetic adsorbent DIAION HP20 (a styrene-divinylbenzene copolymer, manufactured by Mitsubishi Chemical Corporation (“DIAION” is a registered trademark)) and palladium acetate, in accordance with the method of Example 1 of JP 2008-114164 A. The obtained supported metal catalyst was one in which Pd was supported on the synthetic adsorbent (Pd/HP20), and the amount of supported Pd was 9.5 mass % based on the total mass of the supported metal catalyst.

### Example 1

Acetaminophen was synthesized in the flow synthesis system, illustrated in FIG. 2. The reaction vessel used was one in which 2.55 g (amount of supported Pd: 0.24 g (2.3 mmol)) of the Pd/HP20 (produced in Synthesis Example 1) was packed into an SUS column having a size of 10 mm (diameter)×100 mm (length).

1 L of a methanol solution of 0.84 mol/L (13.9 mass %) p-nitrophenol, 500 mL of acetic anhydride, which was used as an acetylating agent, and a hydrogen gas were gradually mixed together, and the mixture was fed into and flowed through the column for 20 minutes in a state in which the column temperature was maintained at 35° C. in a water bath. In this instance, the feed rate of the methanol solution of p-nitrophenol was maintained at 3.6 mL/minute, and the feed rate of the acetic anhydride was maintained at 0.336 mL/minute, with a diaphragm pump and a cylinder pump; and the feed rate of the hydrogen gas was maintained at 240 mL/minute with a mass flow controller. These conditions correspond to an amount of a hydrogen gas fed of 3.6 mol and an amount of acetic anhydride fed of 1.2 mol, per mol of the p-nitrophenol. Furthermore, a Teflon tube and a back-pressure valve were attached to the outlet of the reaction vessel, and the back pressure was set to be 0.5 MPa. Table 3 shows the feed rate of the methanol solution of p-nitrophenol, the reaction time, the reaction pressure, and the reaction temperature.

The obtained reaction product liquid was analyzed with the analysis method 1 and found to contain 8.8 g of acetaminophen (yield: 96.0%).

### Examples 2 to 5

A reaction was performed as in Example 1, except that as opposed to Example 1, the solvent of the p-nitrophenol solution, the reaction pressure, the feed rate, and the reaction time were as shown in Table 3. The obtained reaction product liquid was analyzed as in Example 1, and the results are summarized in Table 3.

### Comparative Examples 1 to 3

A reaction was performed as in Example 1, except that as opposed to Example 1, 4.4 g (amount of supported Pd: 0.24 g (2.3 mmol)) of a catalyst (Pd/C (beads), manufactured by N.E. Chemcat Corporation), which is a catalyst in which Pd is supported on carbon (beads), was used instead of Pd/HP20, and the feed rate of the methanol solution of p-nitrophenol and the reaction time were as shown in Table 3. The obtained reaction product liquid was analyzed as in Example 1, and the results are summarized in Table 3.

Note that using Pd/carbon (powder) instead of Pd/HP20 for comparative examples was considered, but Pd/carbon (beads) was used in the Comparative Examples, because it was surmised that, since Pd/carbon (powder) has a very small particle size, which causes a very high pressure loss, the solution of p-nitrophenol could not be passed through the reaction vessel, and, therefore, the reaction would not proceed, unless a high pressure was used.

TABLE 3

| | | | PNP | | | | Results of analysis of reaction | | | | |
|---|---|---|---|---|---|---|---|---|---|---|---|
| | Supported | | solution | Reaction | Reaction | Reaction | product liquid (yield) | | | | |
| | metal | | feed rate | time | temperature | pressure | PNP | APAP | PAAPA | PNPA | PAP |
| | catalyst | Solvent | [mL/min] | [s] | [° C.] | [MPa] | [%] | [%] | [%] | [%] | [%] |
| Example 1 | Pd/HP20 | MeOH | 3.60 | 0.9 | 35 | 0.5 | 3.24 | 96.0 | 0.74 | 0.00 | 0.00 |
| Example 2 | Pd/HP20 | MeOH | 3.60 | 0.9 | 35 | 0.2 | 12.6 | 86.5 | 0.86 | 0.00 | 0.00 |
| Example 3 | Pd/HP20 | MeOH | 1.80 | 1.9 | 35 | 0.2 | 0.25 | 99.3 | 0.45 | 0.00 | 0.00 |
| Example 4 | Pd/HP20 | AcOH | 3.60 | 0.9 | 35 | 0.5 | 21.7 | 78.3 | 0.00 | 0.00 | 0.00 |
| Example 5 | Pd/HP20 | AcOH | 3.60 | 0.9 | 45 | 0.5 | 20.6 | 79.3 | 0.06 | 0.00 | 0.00 |
| Comparative | Pd/C | MeOH | 3.60 | 0.9 | 35 | 0.5 | 38.3 | 59.9 | 0.90 | 0.91 | 0.00 |
| Example 1 | (beads) | | | | | | | | | | |
| Comparative | Pd/C | MeOH | 1.80 | 1.9 | 35 | 0.5 | 26.0 | 72.5 | 0.97 | 0.62 | 0.00 |
| Example 2 | (beads) | | | | | | | | | | |
| Comparative | Pd/C | MeOH | 0.90 | 3.7 | 35 | 0.5 | 16.3 | 82.7 | 1.03 | 0.00 | 0.00 |
| Example 3 | (beads) | | | | | | | | | | |

In Table 3, Example 1 and Comparative Examples 1 to 3 demonstrate that in the instance where Pd/HP20 is used, acetaminophen can be produced efficiently in a shorter reaction time and with higher selectivity and better yield, than with Pd/C, which is used in the related art.

Examples 2 and 3 demonstrate that, even at a lower reaction pressure, acetaminophen can be produced efficiently with high selectivity and good yield.

Examples 1, 4, and 5 demonstrate that in the instance where the solvent is changed from methanol to acetic acid, formation of PAAPA can be inhibited.

### Examples 6 to 15

A reaction was performed as in Example 1, except that as opposed to Example 1, the size of the column was 10 mm (diameter)×250 mm (length) rather than 10 mm (diameter)×100 mm (length), the amount of use of Pd/HP20 was 6.38 g rather than 2.55 g (the amount of use of Pd/HP20 relative to the volume of the column was unchanged), and the reaction temperature, the reaction pressure, and the feed rate of the methanol solution of p-nitrophenol were as shown in Table 4. The obtained reaction product liquid was analyzed as in Example 1, and the results are summarized in Table 4.

TABLE 4

| | | | PNP | | | | Results of analysis of reaction | | | | |
|---|---|---|---|---|---|---|---|---|---|---|---|
| | Supported | | solution | Reaction | Reaction | Reaction | product liquid (yield) | | | | |
| | metal | | feed rate | time | temperature | pressure | PNP | APAP | PAAPA | PNPA | PAP |
| | catalyst | Solvent | [mL/min] | [s] | [° C.] | [MPa] | [%] | [%] | [%] | [%] | [%] |
| Example 6 | Pd/HP20 | MeOH | 0.38 | 22.3 | 25 | 0.1 | 0.00 | 99.3 | 0.71 | 0.00 | 0.00 |
| Example 7 | Pd/HP20 | MeOH | 0.56 | 14.5 | 25 | 0.1 | 0.00 | 99.0 | 1.04 | 0.00 | 0.00 |
| Example 8 | Pd/HP20 | MeOH | 0.75 | 11.1 | 25 | 0.1 | 0.00 | 98.9 | 1.08 | 0.00 | 0.00 |
| Example 9 | Pd/HP20 | MeOH | 1.13 | 7.4 | 25 | 0.1 | 1.20 | 97.3 | 1.53 | 0.00 | 0.00 |
| Example 10 | Pd/HP20 | MeOH | 1.50 | 5.6 | 25 | 0.1 | 10.3 | 88.2 | 1.50 | 0.00 | 0.00 |
| Example 11 | Pd/HP20 | MeOH | 1.50 | 5.6 | 35 | 0.1 | 0.00 | 98.9 | 1.09 | 0.00 | 0.00 |
| Example 12 | Pd/HP20 | MeOH | 2.25 | 3.7 | 35 | 0.1 | 6.41 | 92.3 | 1.30 | 0.00 | 0.00 |
| Example 13 | Pd/HP20 | MeOH | 2.25 | 3.7 | 45 | 0.1 | 1.47 | 83.9 | 1.04 | 0.00 | 13.56 |
| Example 14 | Pd/HP20 | MeOH | 2.25 | 3.7 | 35 | 0.2 | 0.00 | 98.7 | 1.31 | 0.00 | 0.00 |
| Example 15 | Pd/HP20 | MeOH | 4.50 | 1.9 | 35 | 0.2 | 0.00 | 99.0 | 0.96 | 0.00 | 0.00 |

In Table 4, Examples 6 to 13 demonstrate that even when the reaction pressure is a pressure close to a normal pressure, acetaminophen can be produced efficiently with high selectivity and good yield in the instance in which an appropriate reaction temperature and reaction time are selected.

In Example 15, the length of the column was changed from that of Example 3. From Example 15 and Example 3, it is apparent that comparable results can be obtained even if the length of the column is changed, that is, the results are not affected by the length of the column.

In Example 14, the reaction pressure was increased from that of Example 12. From the results, it is apparent that increasing the pressure increases reactivity.

### INDUSTRIAL APPLICABILITY

The method of the present invention for producing acetaminophen can continuously produce acetaminophen, which is useful as a medicine, from p-nitrophenol, safely and inexpensively, with high selectivity and good yield, under mild conditions of a low reaction temperature and a low reaction pressure, without requiring high-pressure reaction equipment, and, therefore, the method is industrially useful.

Although the present invention has been described in detail with reference to particular embodiments, it will be apparent to those skilled in the art that various changes can be made without departing from the spirit and the scope of the present invention.

The present application is based on Japanese Patent Application No. 2020-086766 filed on May 18, 2020, which is herein incorporated in its entirety by reference.

### REFERENCE SIGNS LIST

1 Supported metal catalyst of present invention

2 Column

3 Reaction vessel

4 Collection reservoir

5 Back-pressure valve

1. A method for producing acetaminophen, the method comprising:
passing a solution comprising p-nitrophenol through a column packed with a catalyst while also passing an acetylating agent and hydrogen through the column, thereby causing p-nitrophenol to undergo an acetamination reaction to produce the acetaminophen,
wherein the catalyst is a supported metal catalyst in which a metal element is supported on a synthetic adsorbent,
wherein a reaction temperature of the acetamination reaction is in a range of from 0° C. to 60° C., and
wherein a reaction pressure of the acetamination reaction is in a range of from 0.1 MPa to 1 MPa.

2. The method of claim 1, wherein the synthetic adsorbent is a styrene-divinylbenzene-based copolymer.

3. The method of claim 2, wherein the styrene-divinylbenzene-based copolymer is a styrene-divinylbenzene copolymer, and
wherein the metal element is palladium and/or platinum.

4. The method of claim 1, wherein the synthetic adsorbent is a porous synthetic adsorbent having a pore volume in a range of from 0.1 mL/g to 3.0 mL/g.

5. The method of claim 1, wherein the synthetic adsorbent is a porous synthetic adsorbent having a BET specific surface area in a range of from 200 m²/g to 2000 m^{2/}g.

6. The method of claim 1, wherein the synthetic adsorbent is a porous synthetic adsorbent having a mode pore radius in a range of from 1 nm to 50 nm.

7. The method of claim 1, wherein an amount of the supported metal element in the supported metal catalyst is in a range of from 1 mass % to 25 mass % based on a mass of the supported metal catalyst.

8. The method of claim 1, wherein the synthetic adsorbent comprises, in copolymerized form, styrene and divinylbenzene.

9. The method of claim 1, wherein the metal element comprises palladium.

10. The method of claim 1, wherein the metal element comprises platinum.

11. The method of claim 1, wherein the metal element comprises palladium and platinum.
